(19) United States
(12) Patent Application Publication
(10) Pub. No.: US 2022/0000773 A1
(43) Pub. Date: Jan. 6, 2022

(54) SELF-REMINDING PATCH

(71) Applicant: LTS Lohmann Therapie-Systeme AG, Andernach (DE)

(72) Inventor: Kenneth Glade, West Milford, NJ (US)

(21) Appl. No.: 16/625,401

(22) PCT Filed: Jun. 26, 2018
(86) PCT No.: PCT/US2018/039579

§ 371 Date: Dec. 20, 2019

Related U.S. Application Data
(60) Provisional application No. 62/525,586, filed on Jun. 27, 2017.
(60) Provisional application No. 62/616,781, filed on Jan. 12, 2018.

(51) Int. Cl. A61K 9/00; A61K 9/70

(52) U.S. Cl. CPC A61K 9/0097; A61K 9/0014; A61K 9/7084

(57) ABSTRACT

The inventive transdermal therapeutic systems (TTS) incorporate a timing device, such as an electronic timing device, particularly a thin, electronic timing device. The timing device can be a part of, adhered to, or used in lieu of the TTS backing layer. The device generally includes a pre-programmed or pre-designated timer, an activation and/or reset button to start or reset the timer and an indicator, such as a visual indicator, to alert the patient when the administration time period has lapsed.

### CROSS REFERENCE TO RELATED APPLICATIONS

This application claims priority to Provisional Application No. 62/616,781, filed Jan. 12, 2018, which is a continuation-in-part application claiming priority to Provisional Application No. 62/525,586, filed Jun. 27, 2017, both of which are hereby incorporated herein in their entirety.

### FIELD OF THE INVENTION

The present invention relates to transdermal therapeutic systems and methods for manufacturing the same. The invention particularly relates to transdermal therapeutic systems which comprise a timing device.

### BACKGROUND OF THE INVENTION

Transdermal therapeutic systems (“TTSs”), also commonly referred to as transdermal patches, are generally known. Transdermal therapeutic systems are multilayered planar devices that administer one or more active agents, particularly one or more pharmaceutical active agents, via a patient's skin. One of the significant benefits of TTS technology is the delivery of active agent to and through the patient's skin at a constant rate over a longer period of time relative to oral medicaments.

Extended active agent delivery, such as provided by a transdermal therapeutic system, is particularly important in the treatment of chronic conditions, such as chronic pain, dementia, Alzheimer's disease and Parkinson's disease. The market for these products is substantial, with patches for Alzheimer's disease having $400 million in annual sales as of 2015, for example. Transdermal therapeutic systems are typically designed to be removed after a single day. In contrast, extended delivery patches, such as patches for chronic pain or conditions, can be worn over a period of at least 72 hours, e.g. Duragesic Transdermal therapeutic systems suitable for long term wear are described in United States Patent Application Publication No. 2012/0157937 and U.S. Pat. No. 8,883,198, both of which are hereby incorporated by reference herein in their entirety.

Unfortunately, existing transdermal patches contain no method within the patch itself to remind the patient or caregiver to remove to replace the patch. For extended delivery transdermal therapeutic systems it is especially difficult to remember when the patch was applied. Some patients, especially patients suffering from Alzheimer's disease or dementia, have extreme difficulty remembering the time of application and/or the prescribed replacement period. This confusion leads to missed doses for patches replaced too late, or overdoses for patches replaced too soon. When patches are replaced too late, or doses entirely missed, the treated condition (such as dementia) can readily return, pain can be improperly managed, and the like. Overdoses due to mistiming are similarly problematic. Regrettably, many of the medicaments used to treat chronic conditions are quite potent, and can further induce a number of side effects, including nausea, vomiting and the like. In addition, one or more caregivers may have responsibility for administering the transdermal patches to one or more patients, particularly patients suffering from Alzheimer's or dementia. In such group settings it can be especially difficult to track patch application dates and times. Estimates of the costs incurred by patience noncompliance range about $ 100 to 290 billion a year.

Recently, the FDA approved the first digital pill, i.e. medication embedded with a sensory, that allows physicians to determine when, or if, patients take their medication. The digital pills are designed so that the doctor and up to four other people can view patient adherence. Currently, the only approved digital pill is used to treat schizophrenia; however, digital pills are being used or tested for patients suffering from heart problems and diabetes, inter alia. The pills sensor, developed by Proteus Digital Health, generates an electrical signal upon contacting stomach fluid. The electrical signal is them detected by a patch which must be applied to the left rib cage and replaced every seven days. Alternate ingestible sensors generate a low power radio signal that can be picked up by a reader worn on the patient's neck or the like.

Printed electronics, such as labels or tags used in tracking durable good within a supply chain are generally known, albeit a developing technology. Transdermal therapeutic systems incorporating radio tags are known, as described in United States Patent Application Publication No. 2016/0220800, hereby incorporated by reference herein in its entirety. Although radio tags may be used to relay information to a separate transponder, the TTS itself does not alert the wearer as to the dosage duration. The use of a separate transponder is especially problematic for patients with memory loss or other cognitive impairment issues, and requires that the receiver device be present.

Thin printed electronic timers are being developed as reminders for furnace filter and smoke detector replacement. Thin printed electronic timers are not known in the art for incorporation within TTSs; however.

Accordingly, a need in the art remains for TTSs incorporating thin, flexible timers that remind patients and caregivers to replace the patch.

### SUMMARY OF ADVANTAGEOUS EMBODIMENTS OF THE INVENTION

The inventive transdermal therapeutic systems incorporate a timing device, preferably an electronic timing device, as a part of, in addition to or in lieu of the TTS backing layer. The timing device generally includes (i) a timer, such as a pre-programmed timer set to the dosage period; (ii) an activation and/or reset button to start or reset the timer and (iii) an indicator, such as a visual indicator, to alert the patient when the time period has lapsed or expired.

### BRIEF DESCRIPTION OF THE DRAWINGS

FIG. 1 is a schematic cross-sectional view of an exemplary transdermal therapeutic system in accordance with the invention;

FIG. 2A is a schematic cross-sectional view of an alternate exemplary transdermal therapeutic system in accordance with the invention;

FIG. 2B is a schematic top-view of the exemplary alternate transdermal therapeutic system of FIG. 2A;

FIG. 3A is a schematic top-view of a further exemplary embodiment of the inventive transdermal therapeutic systems; and

FIG. 3B is a schematic cross-sectional side view of the further exemplary transdermal therapeutic system in accordance with the invention;

FIG. 4A illustrates a cross-section of an exemplary electrophoretic display;

FIG. 4B illustrates an exemplary electrophoretic display with a microcapsule ink layer;

FIG. 4C illustrates an exemplary electrophoretic display with a microcup ink layer;

FIG. 5 illustrates an exemplary timing module based upon an electrophoretic display, including the associated timing electronics;

FIG. 6 provides an exemplary transdermal therapeutic system incorporating an EPD-based timing module;

FIG. 7 illustrates an exemplary transdermal therapeutic system incorporating an EPD-based timing module, as applied to the skin;

FIG. 8A illustrates a first screenshot from an exemplary smartphone application specifically tailored to the inventive transdermal therapeutic systems;

FIG. 8B illustrates a second screenshot from an exemplary smartphone application specifically tailored to the inventive transdermal therapeutic systems proving instructions as to use;

FIG. 9 illustrates an exemplary inventive transdermal therapeutic system incorporating a scannable/enterable bar code, as applied to the skin.

### DETAILED DESCRIPTION OF ADVANTAGEOUS EMBODIMENTS OF THE INVENTION

The present invention is directed to a transdermal therapeutic system that generally includes, in spatial order, a timing and/or monitoring device, one or more optional reservoirs comprising at least one active ingredient, and an optional detachable protective layer.

The timing and/or monitoring device may be any timing device with sufficient size and/or flexibility to be adhered to or enclosed within a transdermal therapeutic system. Exemplary timing devices include printed electronic timing devices, chromatography-based timing devices, and chemical timing devices. Suitable timing device generally include a pre-programmed, pre-set or pre-designated timer set to a dosage period (also referred to herein as “administration period”), an activation and/or reset button, an indicator, such as a visual, audio and/or vibratory indicator and a power source.

Suitable printed electronic timers and/or timing devices, also referred to in the art as printed timer labels, include those described in United States Patent Application Publication No. 2016/0095226, assigned on its face to Thin Film Electronics ASA, Oslo NO, hereby incorporated by reference herein in its entirety. Suitable printed electronic timing devices are also available from RR Donnelley, Chicago Ill. Printed electronic timing devices generally have a flat, planar film-shape; include a timer substrate, a printed battery on the substrate, a printed load resistance and printed voltage comparison circuitry, and indication devices, as known in the art. In alternative embodiments, the printed electronic timer may also connect to a separate battery, either in addition to or in lieu of the printed battery.

Suitable chromatography-based timing devices include TIMESTRIP indicators, from Timestrip UK, Cambridge UK. In chromatograph-based timing devices a colored line appears when the activation button has been pushed, and the color progresses along printed time markers in or alongside the viewing window until a predetermined time, such as 3 days or a week, has been reached. More specifically, in such chromatic timers, a tinted liquid moves through a viewing window, such as a white viewing window formed from a porous substrate, at a pre-calibrated rate. When the color reaches the end of the window, the full predetermined time has elapsed and the transdermal therapeutic system should be removed from the patients' skin. The chromatography-based timing devices are activated by fully squeezing the activation button between a thumb and finger immediately prior to applying the transdermal therapeutic system to the patient's skin. Chromatic timers provide a constant visual reminder of the treatment time remaining without requiring a battery or other electronic components.

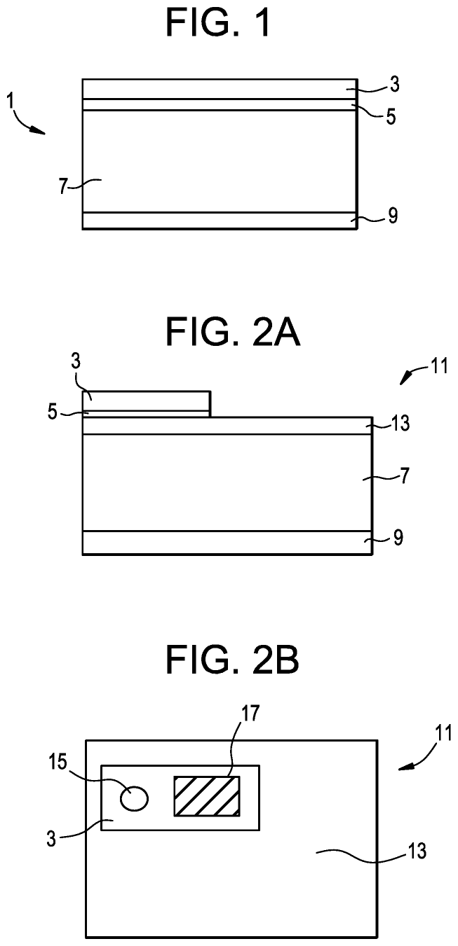

FIGS. 1 through 3 illustrate different exemplary embodiments of the inventive transdermal therapeutic systems. The reference numbers used herein have the following meaning within each of FIGS. 1, 2A and 2B, 3A and 3B:
1=first embodiment of an exemplary inventive transdermal therapeutic system
3=timing and/or monitoring device
5=adhesive layer
7=reservoir layer
9=removable protective layer
11=second embodiment of an exemplary inventive transdermal therapeutic system
13=backing layer
15=activation device
17=indicator

In advantageous embodiments, the timing and/or monitoring device 3 serves as the backing layer of the transdermal therapeutic system, as illustrated in FIG. 1. In alternative embodiments, the transdermal therapeutic system 11 includes a separate backing layer 13 disposed on a surface of the reservoir(s) 7 opposite the optional detachable protective layer 9, and the timing device 3 is adhered to the backing layer 13 via an adhesive layer 5, as illustrated in FIGS. 2A and 2B. The timing device 3 can be adhered to the surface of the backing layer 13 opposing the reservoir(s) 7.

As shown in FIG. 2B, the timing device generally includes at least an activation device 15 and indicator 17.

Alternatively, the timing device may be disposed between the backing layer and reservoir(s). In such alternative embodiments, the backing layer is transparent, such as polyester film or the like, and protects the timing device from water damage. In additional alternative embodiments, an additional protective layer, such as transparent film layer, may be adhered to the surface of the timing device opposite the backing layer.

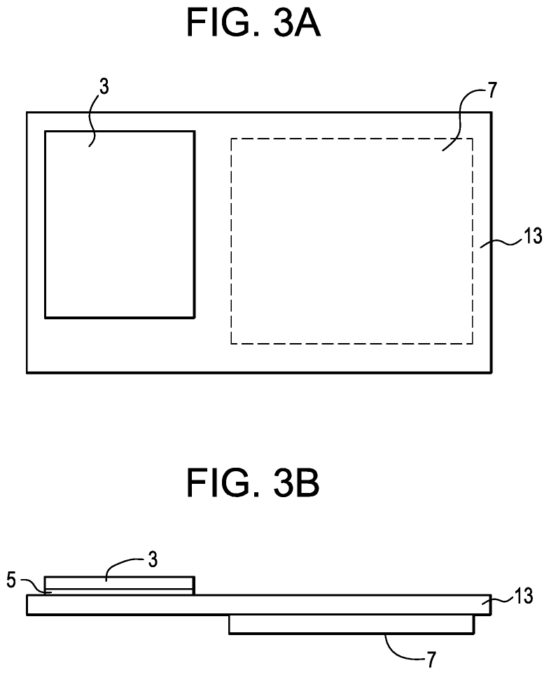

In additional expedient embodiments, the timing and/or monitoring device 3 may be disposed on or adhered to an extension of the backing layer 13 or where there is otherwise no corresponding reservoir (s) 7 or drug layer present, as illustrated in FIGS. 3A and 3B.

The timing and/or monitoring device may be adhered to the transdermal therapeutic system via an adhesive layer comprising any suitable adhesive known in the art, particularly the art of transdermal patches. Suitable adhesives include pressure-sensitive adhesives, such as adhesives formed from polyacrylates, polysiloxane adhesives, polyisobutylene, hot melt adhesives and the like.

The timing and/or monitoring device is expediently adhered to/disposed on at least a portion of the backing layer. The timing and/or monitoring device is generally disposed on from 5 to 100% of the backing layer surface area, for example, such as from 20 to 80% of the backing layer surface area. As noted above, the timing and/or monitoring device may also be used in lieu of a backing layer, as well, as illustrated in FIG. 1. In further alternative embodiments, the timing and/or monitoring device may be encapsulated within a coating formed from the backing layer polymer and/or other water-proof or water-resistant transparent material prior to adhering it to the backing layer.

As noted above, the timing and/or monitoring device further comprises an activation device. Non-limiting exemplary activation devices include one or more of an activation button, pull tag and/or activation pad. As used herein the terms “activation” and “start” may be used interchangeably. The activation device is advantageously triggered, such as by pushing, either immediately prior to or immediately after TTS application onto the patient or user, so that the administration period may be accurately reflected. In expedient aspects, the timing device further comprises a reset and/or stop button or pad, so that the transdermal therapeutic system may be removed and reused if necessary or the like.

The timing and/or monitoring device further comprises an indicator or display to signal or alert the user, patient and/or caregiver that the administration time has lapsed and the transdermal therapeutic system should be removed or replaced. The indicator may be a visual indicator, such as a light or LED screen. When triggered, the indicator may produce a steady light, blinking light, or the LED screen may flash instructional printed indicia such as “please remove patch” or the like. In addition to or alternatively, the indicator may also include components to produce one or more of an auditory or vibratory alarm, which particularly beneficial for sight impaired patients or users.

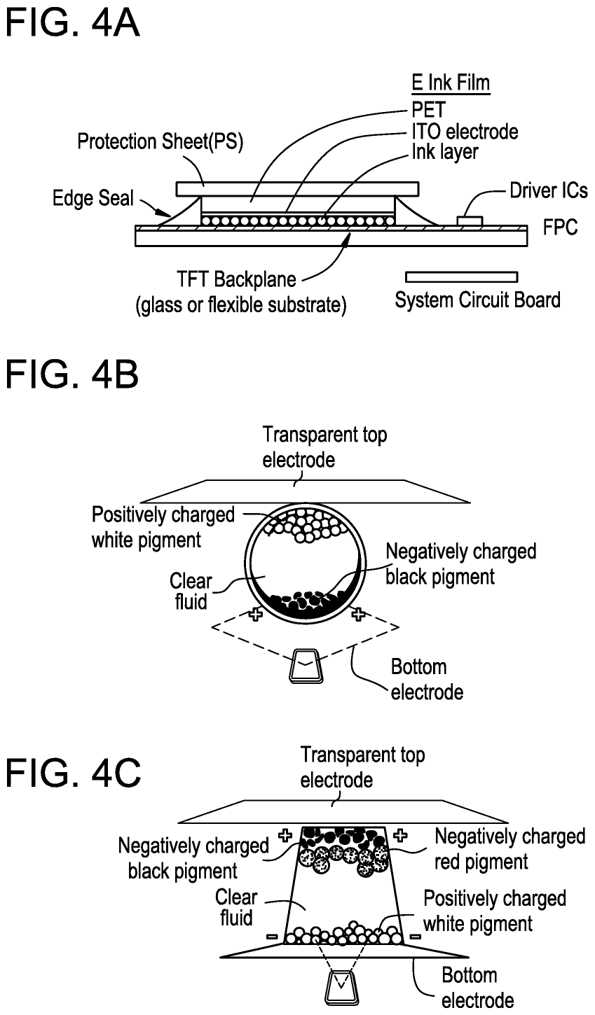

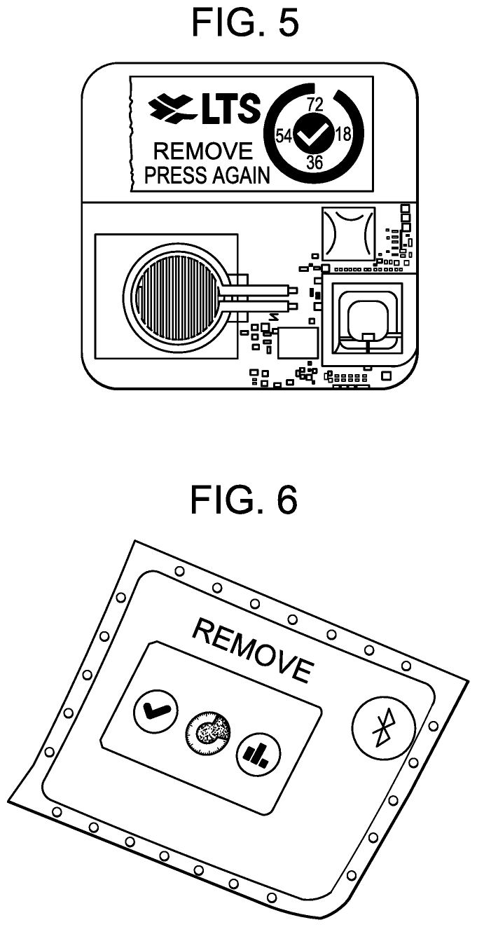

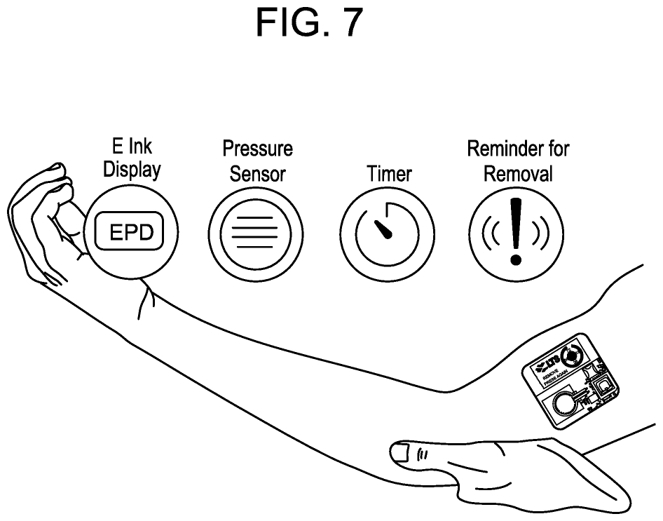

In alternative embodiments, the timing and/or monitoring device may comprise an electrophoretic display (“EPD”) module. As known in the art, EPDs utilize electrophoretic ink, also commonly referred to electronic ink, developed by E Ink Holdings (“E Ink), as illustrated in FIG. 4A. As known in the art, electrophoretic displays, which are also referred to electronic paper displays, can be formed by laminating electronic ink onto a plastic film and adhering the laminate to electronics. Two-pigment electronic ink systems based upon microcapsules, illustrated in FIG. 4B, and three-pigment electronic ink systems based upon a micro-cup structure, illustrated in FIG. 4C, both commercially available from E Ink, are both suitable for use within the inventive transdermal therapeutic systems. TTSs incorporating EPD technology generally include an electrophoretic display, preferably a low voltage electronic ink display, a microcontroller, a switch, pressure sensor and battery. The low voltage electronic ink display or film, commercially available from E Ink, uses 50 to 70% of the typical driving voltage for known EPDs, comprises a small, durable battery with longer battery life, and has a thickness of less than 200 microns. An exemplary EPD module and associated timing electronics are shown in FIG. 5, with an exemplary TTS incorporating such EPD module and timing electronics shown in FIGS. 6 and 7.

Additional optional features which may be included within the inventive TTSs include Bluetooth technology, radio frequency integrated circuit(s) and antenna(s).

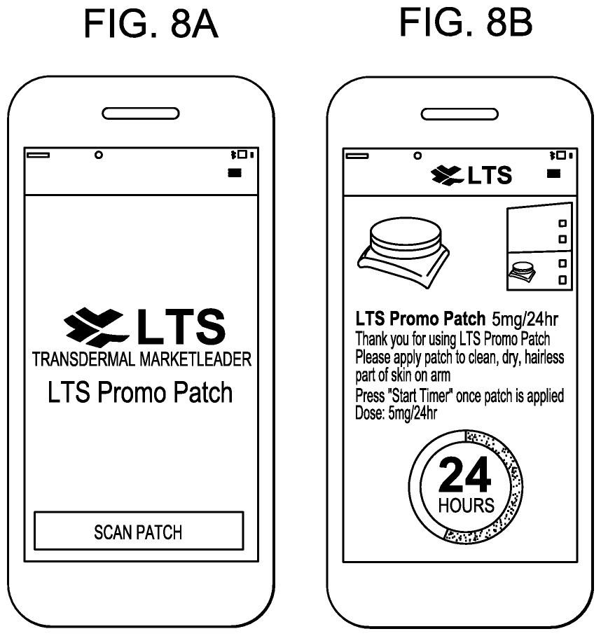

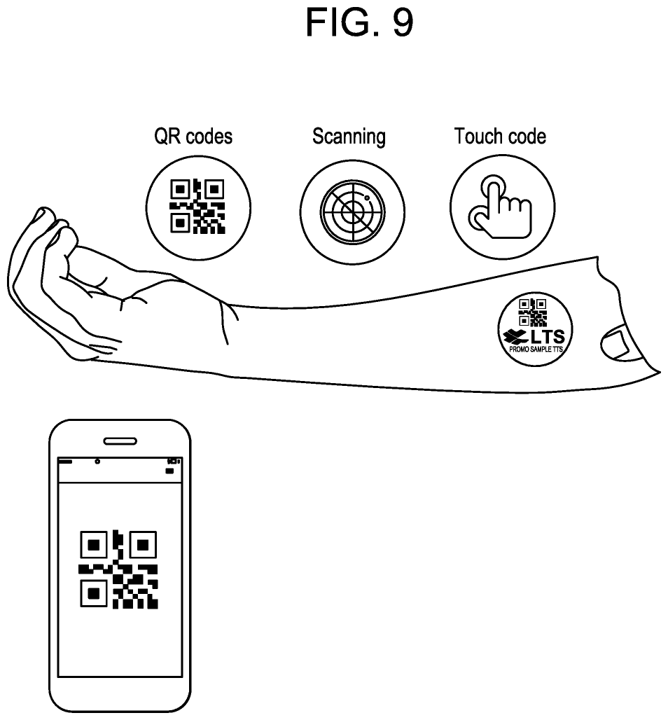

In further embodiments, the transdermal therapeutic system may be connected to a mobile and/or computer application, e.g. through printed electronics or the like, designed to alert the patient or caregiver either of the current administration time remaining or of a lapsed administration time and/or any of a number of other monitored responses, such as vital signs or glucose levels, via a smartphone, tablet, smartwatch and/or computer application or the like. In such expedient embodiments, the inventive transdermal therapeutic system could be used to dovetail with existing tracker applications or new applications could be specifically tailored to the given TTS, as illustrated in FIG. 8A, which provides an exemplary introductory screenshot, and FIG. 8B, which provides an exemplary screenshot with more detailed information. For example, for transdermal therapeutic systems delivering nicotine into the patient's system, a mobile application could be paired with the TTS that provides behavioral support. Alternatively, for transdermal therapeutic systems delivering narcotics such as opioids or other regulated additive substance, the inventive TTS can be designed to emit a signal upon being tampered with and/or stolen. The mobile application may further assist in the daily routines of caregivers, such as by providing easier and faster documentation, feeding patient information automatically into a digital patient record, reminding the caregiver(s) to replace the TTS or buy a new one, automatically reordering/restocking TTS inventory, automatically billing the health insurance company upon TTS replacement. In that regard, each of the inventive TTSs may include a scannable/enterable bar code, such as a quick response (“QR”) code, or other code, that synchronizes the mobile application with the respective individual TTS and/or its production lot, as illustrated in FIG. 9. The incorporation of a scannable code allows for easy positive identification by caregiver(s) of the TTS either before or during patient administration via smart phone or the like, and could further be used to allow ready access to product information, patient history and other pertinent information.

The inventive transdermal therapeutic system and/or the timing and/or monitoring device may further comprise a radio tag, such as either an active or passive RFID transponder, microchip and/or magnetic strip that may provide patient data to the mobile application or hand held transponder. The data may be used in the mobile application to provide any of a number of key features, including one or more of: a calendar providing usage history; tips and alerts, such as information about the product and company; active ingredient facts, photos and informational videos; a link to social media, such as Twitter or various web pages; social media icons, coupons; and full length tutorials.

The timing and/or monitoring device may have any thickness known in the art of timer labels, including thicknesses that are as thin as a layer of printed ink. Non-limiting exemplary thicknesses typically range from about 25 micron to 3 mm, such as from 50 micron to 1 mm, with thinner timing devices being preferable due to their increased flexibility. Non-limiting exemplary thicknesses for electronic paper displays range from 100 to 400 microns, such as less than 200 microns. As used herein, the term “flexibility” refers to the ability of a given layer or device to bend easily when exposed to a small force directed perpendicularly onto the layer. In advantageous embodiments, the overall flexibility of the inventive transdermal therapeutic systems is not substantially impaired by the timing device.

The timing and/or monitoring device may further have any suitable length or width, such as up to the length and width of any the remaining transdermal therapeutic components. Exemplary dimensions for printed electronic timers ranges from about 1 cm by 10 cm, such as from 1.5 cm by 2 cm. Exemplary dimensions for chromatography-based timing devices range from 10 mm by 50 mm, such as 19 mm by 40 mm. Non-limiting exemplary dimensions (i.e. length and/or width) for electronic paper displays range from 1 to 3 inches, such as 2 inches, with the overall module including the electronic paper display ranging from 2 to 10 cm, such as 5.5 cm.

The timing and/or monitoring device may be pre-programmed, pre-designated or otherwise designed to indicate the passage and/or lapse of any administrative time known in the art of transdermal therapeutic systems. As used herein, the term “administrative time” refers to a time that begins upon transdermal therapeutic system application to the patient's skin and ends when the predetermined dosage of active ingredient(s) has been administered. Exemplary administration times for which the timing device may be pre-programmed or designed to indicate can range anywhere from 1 hour to about 168 hours, such as at least 24 hours, preferably at least 48 hours, and particularly preferably at least 72 hours.

Additional exemplary functionalities that could be incorporated include, inter alia, an indication of whether the patch is applied correctly, wearing/application time, a countdown until the next dose, a reminder to remove the patch and/or replace it, and active ingredient content level. The inventive transdermal therapeutic systems may further include functional devices providing one or more of communication (such as connection to a doctor and/or emergency responder), data analysis, monitoring, stimulation or sensors. Inventive transdermal may provide any of a number of monitoring functions, including continuous blood pressure, heart rhythm, heart rate, body temperature, UV exposure and/or glucose monitoring, for example, in lieu or in addition to the delivery of active ingredient(s). The inventive TTS may report the foregoing functionalities, particularly the foregoing monitoring, to a mobile application as described above and/or may display the monitoring/functional results on one or more displays incorporated into the TTS, such as via the EPD technology described above.

The remaining components of the inventive transdermal therapeutic systems, i.e. the optional backing layer, active ingredient(s) reservoir (which is optional in purely monitoring embodiments), optional membranes and optional removable protective layer, can be any of the respective components known in the art of transdermal therapeutic systems. For point of reference, as used herein the backing layer is located on the side facing away from the patient or user, while the optional removable protective layer is located on the transdermal therapeutic system side that will face the patient or user, as is well known in the art.

The backing layer is typically impermeable to the active ingredient(s), thereby preventing the active ingredient(s) from passing out through the backing layer. The backing layer may be permeable to moisture, by which is meant that the backing layer is permeable to water vapor and/or water in liquid form. In a preferred embodiment; the backing layer is impermeable to water in liquid form. The backing layer and/or optional protective layer is generally formed from one or more of a fabric, film, paper or metal-paper composite. Exemplary fabrics and films may be formed from any natural, synthetic or regenerated polymer known in the art. Suitable polymeric films and fabrics include any known polyester film or fabric, particularly polyethylene terephthalate film or fabric. Especially suitable backing layer constructions for long term administration are provided in United States Patent Application Publication No. 2012/0157937. As used herein, the term fabric includes woven, knit and non-woven fabrics. Non-limiting exemplary backing layer surface areas include any surface area known in the art for conventional transdermal therapeutic systems, including from 1 to 100 cm², such as from 10 to 30 cm².

The active ingredient(s) reservoir may be any suitable active ingredient reservoir known in the art of transdermal therapeutic systems. As used herein, the term “reservoir” may refer any drug containing area and/or layer. Exemplary reservoirs include encapsulated reservoirs having an active-ingredient permeable membrane facing the skin and matrix-based reservoirs. Suitable matrix-based reservoirs include drug-in-adhesive reservoirs, matrix-dispersion systems, and micro-reservoir systems, all of which are known in the art, and which may likewise include an active-ingredient permeable membrane disposed between the active-ingredient containing layer (e.g. drug-in-adhesive layer, matrix-layer or micro-reservoir layer) and the optional removable protective layer. In drug-in-adhesive reservoirs and matrix-dispersion systems, the reservoir is formed from a polymer matrix in which the active ingredient is dissolved or suspended. Drug-in-adhesive reservoirs are formed by dispersing the active ingredient in an adhesive polymer to form a reservoir composition and then applying the reservoir composition by casting or coating one or more active-ingredient-containing reservoir layer(s) onto either the backing layer and/or the timing device (for embodiments in which the timing device replaces the backing layer). Further layers of unmedicated adhesive may optionally be applied onto at least a portion the surface of the adhesive-active-ingredient layer(s) facing the optional removable protective layer, or a layer of unmedicated adhesive may be present between the reservoir layer(s) and the backing layer. In advantageous embodiments, the active-ingredient and/or unmedicated adhesive polymer(s) is self-adhesive polymer.

Matrix-dispersion systems disperse or dissolve the active ingredient(s) in a hydrophilic or lipophilic polymer matrix. The active-ingredient containing matrix is formed into one or more reservoir layer(s), such as disk-shape reservoir layer(s), via casting coating or the like. The reservoir layer is then adhered to the backing layer, and adhesive, such as self-adhesive polymer, is applied onto either (i) at least a portion of the surface of the reservoir layer(s) facing the optional removable protective layer and/or (ii) the backing layer (or timing device) behind and/or annularly extending past the circumference of the reservoir layer. In matrix-dispersion embodiments, the transdermal therapeutic systems thus include a separate self-adhesive layer either disposed on or covering at least a portion of the outermost reservoir layer(s) or disposed on the backing layer as an annular ring extending past the reservoir perimeter or circumference.

Micro-reservoir systems are formed by first suspending the drug in an aqueous solution of water-soluble polymer and then dispersing the solution homogeneously in a lipophilic polymer matrix to form reservoir compositions containing dispersed, roughly spherical shaped drug reservoirs within the polymer matrix. The lipohilic polymer matrix may then be crosslinked to stabilize the reservoir composition, preferably subsequent to the composition having been formed into a reservoir layer. Micro-reservoir embodiments of the transdermal therapeutic systems may further include a self-adhesive layer disposed on and covering at least a portion of one or both of the reservoir layer surfaces or disposed on the backing layer such that an annular ring of self-adhesive extending past the reservoir perimeter or circumference is present.

Any polymer matrix known in the art of transdermal therapeutic systems may be used within the active ingredient reservoir and/or any optional adhesive layer, including one or more of water-repelling polymers, water-swellable polymers, water-soluble polymers and water vapor-permeable polymers. Non-limiting exemplary polymers suitable as matrix polymers include one or more of cross-linked poly(ethylene glycol) networks, acrylic-acid matrices, ethyl cellulose, polyvinylpyrrolidone, hydroxypropyl methylcellulose, organogels and pressure sensitive adhesive(s). Non-limiting exemplary pressure sensitive adhesive(s) include any pressure sensitive adhesive known in the art of transdermal therapeutic systems, including polyisobutylenes, polyacrylates, polysiloxanes and hot melt pressure sensitive adhesives As used herein, the term “pressure sensitive adhesive” refers to a material in the dry form and at room temperature, e.g. 23° C., having a permanent initial tack which makes the material able to adhere firmly to a large number of different materials without the pressure required for this being more than can be exerted with a finger.

Any active ingredient known in the art of transdermal therapeutic system as active pharmaceutical or cosmetic ingredients may be incorporated into the inventive TTSs. Non-limiting exemplary active ingredients include one or more of slimming agents, appetite suppressants, therapeutic agents for acidosis, Alzheimer's drugs, analeptics, antihypoxemics, analgesics, antirheumatics, anthelmintics, antiallergics, antianemics, antiarrhythmics, antibiotics, antiinfectives, antidementia drugs (nootropics), antidiabetics, antidotes, antieetics, antivertigo drugs, antieplipetics, antihemorrhagics (antifibrionlytics), antihypertensives, antihypoglycemics, antihypotensives, anticoagulants, antimycotics, antiparasitic drugs, anti-inflammatory drugs, antitussives, expectorants, arterioschlerosis drugs, balneotherapeutic agents and agents for thermotherapy, beta-receptor blockers, calcium channel blockers, inhibitors of the renin-angiotensin system, bronchodilators, antiasthmatics, cholagogue, biliary therapeutic agents, cholinergics, corticoids, dermatologicals, disinfectants, antiseptics, dietetic agents, alimentary therapeutic agents, diuretics, blood flow-stimulating agents, anticraving drugs, enzyme inhibitors, enzyme preparations, transport proteins, fibrinolytics, geriatric drugs, antigout drugs, drugs for influenza infections and colds (influenza remedies), gynecologicals, hemorrhoid remedies (proctologicals), hepatic drugs, hypnotics, sedatives, pituitary hormones, hypothalamus hormones, regulatory peptides and their inhibitors, immunotherapeutic agents, cytokines, cardiac drugs, caries remedies, periodontosis remedies, coronary drugs, laxatives, lipid-lowering agents, local anesthetics, neurotherapeutic agents, gastrointestinal drugs, migraine remedies, minerals, muscle relaxants, anesthetics, parathyroid hormones, calcium metabolism regulators, osteoporosis remedies, neuropathy products, neurotropic agents, ophthalmologicals, otologicals, anti-parkinson drugs, drugs for extrapyramidal disorders, psychoactive drugs, rhinologicals, sinusitis remedies, roborants, tonics, thyroid therapeutic agents, sera, immunoglulins and vaccines, sex hormones and their inhibitors, spasmolytics, platelet aggregation inhibitors, anti-tuberculosis drugs, stimulants, urologicals, vein therapeutic agents, vitamins, wound-treatment agents, cytostatics and metastasis inhibitors.

These include inter alia, beta-estradiol, norethisterone, physostigmine, norelgestromin, norethisterone acetate, nitroglycerin, nicotine, clonidine, moxonidine, fentanyl, testosterone, buprenorphine, galanthamine, morphine, diamorphine, buprion, sildenafil, (−)-5,6,7,8-tetrahydro-6-[propyl[2-(2-thienyl)ethyl]amino]-1-naphthol (“rotigotine”), methylphenidate, ethinylestradiol, and (S)-N-ethyl-3-[1-dimethylamino)ethyl]-N-methyl-phenyl-carbamate (“rivastigmine”). In particularly advantageous embodiments, the active ingredient is an Alzheimer's drug or dementia drug, particularly rivastigmine.

The at least one active ingredient is present in the reservoir in an amount sufficient for the active ingredient to be delivered in an effective amount to the blood circulation over the intended administration period (period of use), which is preferably an extended period of administration. The content of the at least one active ingredient in the reservoir layer generally ranges from between 0.5 to 45% by weight, such as between 3 and 25% by weight, based upon the weight of the reservoir layer.

Non-limiting exemplary administration periods can range from 1 hour to about 168 hours, such as at least 24 hours, preferably at least 48 hours, and particularly preferably at least 72 hours, up to 2 weeks.

Further additives known in the art of transdermal therapeutic systems may be incorporated into any of the various transdermal therapeutic system layers. Tackifiers, permeation enhancers, flow modifiers and the like may be incorporated into the polymer reservoir and/or any of the adhesives described herein.

Additional layers known in the art of transdermal therapeutic systems may also be incorporated. For example, membrane layers may be incorporated between the reservoir layer and the optional detachable protective layer. As known in the art such membrane layers are permeable to the active ingredient(s), thereby allowing the active ingredient to diffuse therethrough at a finite, controllable rate. Suitable membrane layers may be formed from any polymer known in the art of patch membrane formation, including ethylene vinyl acetate, microporous polyethylene, microporous polypropylene, polyethylene, silicone rubber and polyurethane. The membrane layer may further include an adhesive coating, such as a pressure sensitive adhesive coating, on the surface adjacent the optional detachable protective layer, as is known in the art.

The optional removable (which is also referred to herein as “detachable”) protective layer is impermeable to the active ingredient(s), thereby preventing the active ingredient(s) from passing out through the protective layer. The protective layer is generally formed from one or more of a film, paper or metal-paper composite. Exemplary films may be formed from any natural, synthetic or regenerated polymer known in the art. Suitable polymeric films and fabrics include any known polyester film, particularly polyethylene terephthalate film. The protective layer may further incorporate a release coating disposed on the protective layer surface opposite the user, i.e. on the surface adjacent the reservoir or adhesive layer. Suitable release coatings include any silicone coating known in the art of release films.

The inventive transdermal therapeutic systems have any size known in the art for transdermal therapeutic systems. Non-limiting exemplary transdermal therapeutic system surface areas (as well as backing layer surface areas) generally range in size from 1 to 100 cm², such as from 10 to 30 cm². Non-limiting exemplary transdermal therapeutic system thicknesses range from 12 micron to 3 mm, such as 50 micron to 1 mm.

The transdermal therapeutic systems may be formed using methods known in the art of transdermal therapeutic systems.

A non-limiting exemplary process for producing the inventive transdermal therapeutic systems includes forming a reservoir composition by homogeneously incorporating an effective amount of active ingredient into pressure sensitive adhesive; disposing the reservoir composition comprising at least one active ingredient onto either a removable protective layer or carrier layer via any extrusion or coating technology known in the art of transdermal therapeutic systems, thereby forming a reservoir layer. The carrier layer may be formed from any material known in the art, including a film (e.g. a release film), paper, metal-paper composite or any other foraminous surface known in the art, which may further include a release coating, as well. The reservoir layer may optionally be at least partially dried. Either a backing layer or timing and/or monitoring device may then be applied onto a surface of the at least partially dried reservoir layer opposing the detachable protective layer or carrier layer. If applying a backing layer, then a timing and/or monitoring device may be adhered onto at least a portion of the backing layer surface opposing the active ingredient reservoir. The timing and/or monitoring device may be adhered to the outermost surface of the backing layer using any adhesive known in the art, including pressure sensitive adhesive. The pressure sensitive adhesive may be applied by either the timing device manufacturer or the transdermal therapeutic system manufacturer. If adhesive is applied by the TTS manufacturer, then the inventive methods further include applying adhesive onto the timing and/or monitoring device on a side opposing the activation button prior to bonding the timing device to the transdermal therapeutic system backing layer. As noted above, in particularly expedient embodiments, the timing and/or monitoring device may be used in lieu of a backing layer, such that no additional backing layer is required.

In alternative embodiments, the timing and/or monitoring device may be adhered to the backing layer of a transdermal therapeutic system formed using any means known in the art, particularly adhered immediately prior to individually packaging the transdermal therapeutic system. In further alternative aspects, a kit may be provided that includes a number of individually packaged transdermal therapeutic systems along with a number of self-adhesive timers and/or monitors.

The inventive methods of treatment, such as methods of treating chronic conditions, pain or illness, generally includes applying an inventive transdermal therapeutic system to the skin of a patient or user and activating the timing device to start a preprogrammed, predesignated or predetermined time period of administration either immediately preceding transdermal therapeutic system application (such as immediately prior to removing the optional protective layer) or immediately thereafter. Allowing the transdermal therapeutic system to remain on the skin of the user or patient until the timing device indicator flashes, alarms, vibrates or otherwise indicates that the period of administration (which typically ranges from 1 hour to 7 days) has concluded, at which time the transdermal therapeutic system is removed from the skin. Inventive monitoring methods, which optionally occur simultaneously with the inventive treatment, likewise generally includes applying the inventive transdermal therapeutic system to the skin of a patient or user and activating the monitoring device. The transdermal therapeutic system is then allowed to remain on the skin of the user or patient until the conclusion of a predetermined monitoring period, at which time the monitoring device indicator may optionally flash, alarm, vibrate or otherwise indicate that the monitoring period has concluded, at which time the transdermal therapeutic system is removed from the skin.

As used herein, the term “transdermal” means the percutaneous delivery of at least one active pharmaceutical active ingredient to a user (also referred to herein as a “patient”), with at least one active pharmaceutical ingredient being delivered from a transdermal therapeutic system to the surface of the user's unbroken or ablated skin and migrating through the various layers of skin (e.g. stratum corneous, dermis, cutis and subcutis) located underneath the site of application of the transdermal therapeutic system until it is taken up by the underlying blood vessels. For ease of discussion herein, the term “transdermal therapeutic device” can also include devices which monitor a user or patient's well being, such a vital signs, blood glucose level, UV exposure or the like, either with or without delivering active pharmaceutical active agent.

As used herein, the term “patient” and “user” may be used interchangeably.

Additional advantages, features and modifications will readily occur to those skilled in the art. Therefore, the invention in its broader aspects is not limited to the specific details, and representative devices, shown and described herein. Accordingly, various modifications may be made without departing from the spirit or scope of the general inventive concept as defined by the appended claims and their equivalents.

As used herein and in the following claims, articles such as “the”, “a” and “an” can connote the singular or plural.

All ranges noted herein include all values and/or integers subsumed therein to at least the hundredth place.

By way of summary, the present invention thus generally concerns:

(1). A transdermal therapeutic system comprising in spatial order
(a) a timing device,
(b) a reservoir comprising at least one active ingredient, and
(c) an optional detachable protective layer.

(2) A transdermal therapeutic system as described in (1) further comprising a backing layer disposed between the timing device and the active-ingredient reservoir.

(3) A transdermal therapeutic system as described (1) or (2), wherein said transdermal therapeutic system further comprises an adhesive layer bonding the timing device to at least a portion of the backing layer surface opposing the reservoir.

(4) A transdermal therapeutic system as described in (1) to (3), wherein the timing device covers from 5 to 100% of the backing layer surface area.

(5) A transdermal therapeutic system as described in (1) to (4), wherein the timing device comprises (i) a pre-programmed or pre-designated timer set to a dosage period, (ii) an activation button, (iii) an indicator selected from a visual and/or audio indicator and (iv) a power source.

(6) A transdermal therapeutic system as described in (1) to (5), wherein the timing device comprises an indicator consisting of a visual indicator.

(7) A transdermal therapeutic system as described in (1) to (6), wherein the visual indicator is a light or LED screen or electrophoretic display.

(8) A transdermal therapeutic system as described in (1) to (7), wherein the timing device is pre-programmed or pre-designated for an administrative time ranging from 1 hour to 1 week.

(9) A transdermal therapeutic system as described in claims (1) to (8), wherein the timing device further comprises a RFID chip, microchip and/or magnetic strip.

(10) A transdermal therapeutic system as described in (1) to (9), wherein the backing layer and/or optional protective layer comprises one or more of a fabric, film, paper or metal-paper composite.

(11) The transdermal therapeutic system as described in (1) to (10), wherein the reservoir comprises a polymer matrix in which the active ingredient is dissolved or suspended.

(12) The transdermal therapeutic system as described in (1) to (11), wherein the polymer matrix is a self-adhesive polymer.

(13) The transdermal therapeutic system as described in (1) to (12), wherein the transdermal therapeutic system further comprises a self-adhesive layer disposed on and covering at least a portion of the reservoir surface opposing the timing device.

(14) A transdermal therapeutic system as described in (1) to (13), wherein said active ingredient is selected from one or more of slimming agents, appetite suppressants, therapeutic agents for acidosis, Alzheimer's drugs, analeptics, antihypoxemics, analgesics, antirheumatics, anthelmintics, antiallergics, antianemics, antiarrhythmics, antibiotics, antiinfectives, antidementia drugs (nootropics), antidiabetics, antidotes, antieetics, antivertigo drugs, antieplipetics, antihemorrhagics (antifibrionlytics), antihypertensives, antihypoglycemics, antihypotensives, anticoagulants, antimycotics, antiparasitic drugs, anti-inflammatory drugs, antitussives, expectorants, arterioschlerosis drugs, balneotherapeutic agents and agents for thermotherapy, beta-receptor blockers, calcium channel blockers, inhibitors of the renin-angiotensin system, bronchodilators, antiasthmatics, cholagogue, biliary therapeutic agents, cholinergics, corticoids, dermatological s, disinfectants, antiseptics, dietetic agents, alimentary therapeutic agents, diuretics, blood flow-stimulating agents, anticraving drugs, enzyme inhibitors, enzyme preparations, transport proteins, fibrinolytics, geriatric drugs, antigout drugs, drugs for influenza infections and colds (influenza remedies), gynecological s, hemorrhoid remedies (proctological s), hepatic drugs, hypnotics, sedatives, pituitary hormones, hypothalamus hormones, regulatory peptides and their inhibitors, immunotherapeutic agents, cytokines, cardiac drugs, caries remedies, periodontosis remedies, coronary drugs, laxatives, lipid-lowering agents, local anestetics, neurotherapeutic agents, gastrointestinal drugs, migraine rememdies, minerals, muscle relaxants, anesthetics, parathyroid hormones, calcium metabolism regulators, osteoporosis remedies, neuropathy products, neurotropic agents, ophthalmologicals, otologicals, anti-parkinson drugs, drugs for extrapyramidal disorders, psychoactive drugs, rhinologicals, sinusitis remedies, roborants, tonics, thyroid therapeutic agents, sera, immunoglulins and vaccines, sex hormones and their inhibitors, spasmolytics, platelet aggregation inhibitors, anti-tuberculosis drugs, stimulants, urologicals, vein therapeutic agents, vitamins, wound-treatment agents, cytostatics and metastasis inhibitors.

(15) A transdermal therapeutic system as described in (1) to (14), in which the transdermal therapeutic system further comprises a barcode.

(16) A transdermal therapeutic monitoring system comprising in spatial order
(a) a monitoring device,
(b) an adhesive layer, and
(c) an optional detachable protective layer.

(17) A transdermal therapeutic monitoring system as described in (1) to (16) further comprising active ingredient.

(18) A process for forming a transdermal therapeutic system as described in (1) to (17) comprising

forming a reservoir composition by admixing a polymer matrix and at least one active ingredient;

coating or extruding the reservoir composition onto either a detachable protective layer or carrier layer, thereby forming a reservoir layer,

optionally drying the reservoir layer,

disposing either (i) a backing layer or (ii) timing and/or monitoring device onto the optionally dried reservoir layer surface opposite the detachable protective layer or carrier layer; and

if the backing layer was applied, then adhering a timing and/or monitoring device comprising an activation button onto the backing layer surface opposite the reservoir layer.

(19) A process as described in (18), wherein said method further comprises applying adhesive onto the timing and/or monitoring device on a side opposing the activation button.

(20) A process as described in (18) or (19) comprising the timing device and no backing layer.

(21) A method of treating chronic illness comprising

applying a transdermal therapeutic system as described in (1) to (16) for an administration period ranging from 1 hour to 7 days,

activating the timing and/or monitoring device by pushing an activation button to start a preprogrammed or predesignated time period for administration,

optionally checking the visual indicator periodically to determine if the time period has elapsed, and

removing the transdermal therapeutic system when the indicator signals the end of the preprogrammed or predesignated time period.

(22) A method as described in claim 20), wherein the end of the preprogrammed time period is signaled by a blinking light.

(23) A process for monitoring a patient's functions comprising
applying transdermal therapeutic system comprising
   (a) an activatable electrophoretic module,
   (b) an adhesive layer, and
   (c) an optional detachable protective layer; and
activating the electrophoretic module.

(24) A method of treatment as described in (23), in which the transdermal therapeutic system further comprises a barcode and said process further comprises scanning said barcode.

1. A transdermal therapeutic system comprising in spatial order
(a) a timing device,
(b) a reservoir comprising at least one active ingredient, and
(c) an optional detachable protective layer.

2. A transdermal therapeutic system according to claim 1, further comprising a backing layer disposed between the timing device and the active-ingredient reservoir.

3. A transdermal therapeutic system as claimed in claim 2, wherein said transdermal therapeutic system further comprises an adhesive layer bonding the timing device to at least a portion of the backing layer surface opposing the reservoir.

4. A transdermal therapeutic system as claimed in claim 3, wherein the timing device covers from 20 to 80% of the backing layer surface area.

5. A transdermal therapeutic system as claimed in claim 1, wherein the timing device comprises (i) a pre-programmed or pre-designated timer set to a dosage period, (ii) an activation button, (iii) an indicator selected from a visual and/or audio indicator and (iv) a power source.

6. A transdermal therapeutic system as claimed in claim 5, wherein the g device comprises an indicator consisting of a visual indicator.

7. A transdermal therapeutic system as claimed in claim 6, wherein the visual indicator is a light, LED screen or electrophoretic display.

8. A transdermal therapeutic system as claimed in claim 1, wherein the timing device is pre-programmed or pre-designated for an administrative time ranging from 1 hour to 1 week.

9. A transdermal therapeutic system as claimed in claim 1, wherein the timing device further comprises a RFID chip, microchip and/or magnetic strip.

10. A transdermal therapeutic system as claimed in claim 1, wherein the backing layer and/or optional protective layer comprises one or more of a fabric, film, paper or metal-paper composite.

11. The transdermal therapeutic system according to claim 1, wherein the reservoir comprises a polymer matrix in which the active ingredient is dissolved or suspended.

12. The transdermal therapeutic system according to claim 11, wherein the polymer matrix is a self-adhesive polymer.

13. The transdermal therapeutic system according to claim 1, wherein the transdermal therapeutic system further comprises a self-adhesive layer disposed on and covering at least a portion of the reservoir surface opposing the timing device.

14. A transdermal therapeutic system according to claim 1, wherein said active ingredient is selected from one or more of slimming agents, appetite suppressants, therapeutic agents for acidosis, Alzheimer's drugs, analeptics, antihypoxemics, analgesics, antirheumatics, anthelmintics, antiallergics, antianemics, antiarrhythmics, antibiotics, antiinfectives, antidementia drugs (nootropics), antidiabetics, antidotes, antieetics, antivertigo drugs, antieplipetics, antihemorrhagies antihypertensives, antihypoglycemics, antihypotensives, anticoagulants, antimycotics, antiparasitic drugs, anti-inflammatory drugs, antitussives, expectorants, arterioschlerosis drugs, balneotherapeutic agents and agents for thermotherapy, beta-receptor blockers, calcium channel blockers, inhibitors of the renin-angiotensin system, bronchodilators, antiasthmatics, cholagogue, biliary therapeutic agents, cholinergics, corticoids, dermatologicals, disinfectants, antiseptics, dietetic agents, alimentary therapeutic agents, diuretics, blood flow-stimulating agents, anticraving drugs, enzyme inhibitors, enzyme preparations, transport proteins, fibrinolytics, geriatric drugs, antigout drugs, drugs for influenza infections and colds (influenza remedies), gynecologicals, hemorrhoid remedies (proctologicals), hepatic drugs, hypnotics, sedatives, pituitary hormones, hypothalamus hormones, regulatory peptides and their inhibitors, immunotherapeutic agents, cytokines, cardiac drugs, caries remedies, periodontosis remedies, coronary drugs, laxatives, lipid-lowering agents, local anestetics, neurotherapeutic agents, gastrointestinal drugs, migraine rememdies, minerals, muscle relaxants, anesthetics, parathyroid hormones, calcium metabolism regulators, osteoporosis remedies, neuropathy products, neurotropic agents, ophthalmologicals, otologicals, anti-parkinson drugs, drugs for extrapyramidal disorders, psychoactive drugs, rhinologicals, sinusitis remedies, roborants, tonics, thyroid therapeutic agents, sera, immunoglulins and vaccines, sex hormones and their inhibitors, spasmolytics, platelet aggregation inhibitors, anti-tuberculosis drugs, stimulants, urologicals, vein therapeutic agents, vitamins, wound-treatment agents, cytostatics and metastasis inhibitors.

15. A transdermal therapeutic system according to claim 1, wherein said transdermal therapeutic system further comprises a barcode.

16. A transdermal therapeutic monitoring system comprising in spatial order
(a) a timing and/or monitoring device,
(b) an adhesive layer, and
(c) an optional detachable protective layer.

17. A process for forming a transdermal therapeutic system according to claim 1 comprising
forming a reservoir composition by admixing a polymer matrix and at least one active ingredient;
coating or extruding the reservoir composition onto either a detachable protective layer or carrier layer, thereby forming a reservoir layer,
optionally drying the reservoir layer,
disposing either (i) a backing layer or (ii) a timing and/or monitoring device onto the optionally dried reservoir layer surface opposite the detachable protective layer or carrier layer; and
if the backing layer was applied, then adhering a timing and/or monitoring device comprising an activation button onto the backing layer surface opposite the reservoir layer.

18. A process as claimed in claim 17, wherein said method further comprises applying adhesive onto the timing and/or monitoring device on a side opposing the activation button.

19. A process as claimed in claim 18, comprising the timing and/or monitoring device and no backing layer.

20. A method of treating chronic illness comprising applying a transdermal therapeutic system as claimed in claim 1 for an administration period ranging from 1 hour to 7 days,
activating the timing device by pushing an activation button to start a preprogrammed or predesignated time period for administration,
optionally checking the visual indicator periodically to determine if the time period has elapsed, and
removing the transdermal therapeutic system when the indicator signals the end of the preprogrammed or predesignated time period.

21. A method as claimed in claim 20, wherein the end of the preprogrammed time period is signaled by a blinking light.

22. A process for monitoring a patient's functions comprising
applying transdermal therapeutic system comprising
(a) an activatable electrophoretic module,
(b) an adhesive layer, and
(c) an optional detachable protective layer; and
activating the electrophoretic module.

23. A method of treatment as claimed in claim 22, wherein said transdermal therapeutic system further comprises a barcode and said process further comprises scanning said barcode.
